(19) United States
(12) Patent Application Publication
(10) Pub. No.: US 2017/0143768 A1
(43) Pub. Date: May 25, 2017

(54) OINTMENT FOR THE TREATMENT OF HEMORRHOIDAL DISEASE

(71) Applicant: Global Treat SRL, Jud Galati (RO)

(72) Inventor: Mariana LISINSCHI, Galati (RO)

(73) Assignee: GLOBAL TREAT SRL, Jud Galati (RO)

(21) Appl. No.: 15/315,193

(22) PCT Filed: Apr. 24, 2015
(86) PCT No.: PCT/RO2015/000009

§ 371 Date: Nov. 30, 2016

(30) Foreign Application Priority Data
Apr. 28, 2014 (RO) A 2014 00327

(51) Int. Cl. A61K 35/35; A61K 47/44; A61K 36/61; A61K 33/10; A61K 47/10; A61K 47/06; A61K 9/06; A61K 31/125

(52) U.S. Cl. CPC A61K 35/35; A61K 9/06; A61K 47/44; A61K 31/125; A61K 33/10; A61K 47/10; A61K 47/06; A61K 36/61

(57) ABSTRACT

An ointment for the treatment of internal hemorrhoids, able to avoid the advanced stages of the disease and the need for surgical intervention on long term. The ointment for the treatment of hemorrhoidal disease, especially for treating internal hemorrhoids grades I and II has adeps suillus (axungia), lanolin, white vaseline, camphor, calcium carbonate, eucalyptus oil, ethanol (ethyl alcohol) and preservatives. The ointment implementation and testing were structured in two phases, namely: a product design phase and a test phase (the testing was performed at the Faculty of Medicine and Pharmacy of the University “Dunarea de Jos”). In the first phase, it was conducted the biochemical microbiological and functional characterization of the product and in the second phase, it was conducted the selection of the group of patients, the clinical and para-clinical assessment of patients, in order to exclude possible pathologies caused or contributing to hemorrhoidal disease.

The invention relates to an ointment for the treatment of hemorrhoidal disease, in particular for the treatment of internal haemorrhoids, grades I and II.

### TECHNICAL FIELD

Anal-perineal pathology includes disorders with widespread (even social), apparently trivial, but important and responsible for suffering, with major potential for evolution into complications and frightening, even debilitating sequelae, relates to an ointment for the treatment of the hemorrhoidal disease, in particular for the treatment of internal hemorrhoids, grades I and II.

Hemorrhoidal disease affects equally both sexes, with a slight predominance for males. The peak incidence of the disease is recorded between 30-60 years.

As it is known, the mucosa of the anal canal is surrounded by two muscular cylinders, placed one inside the other. First cylinder—the internal sphincter—results in thickening and lengthening of the lower internal circular smooth muscle blanket of rectum; external cylinder is the external sphincter, which also consists of circular but striated muscle fibers (therefore being a voluntary muscle) and contains the fibers of the levator ani (pubo-rectalis) in its deep portion, which downwardly constitutes the surface portion which completely surrounds the internal sphincter and continues with subcutaneous portion, the most superficial, directly related to the anus.

Submucosal space is located between the mucosa of the anal canal and the internal sphincter, representing the area which develops internal hemorrhoids.

Venous circulation of the anal canal is represented by multi-anastomotic, submucosal, trans- and peri-sphincterial venous plexus, plexus performing two venous streams, one superior and one inferior to the pecten.

At the level of internal or submucosal hemorrhoidal plexus, normally there are venous ectasia called ampoules Duret, which constitutes “the physiological hemorrhoids”, having the role of sealing the anal canal opening and closing; these dilatations become pathological entity when are clinically manifesting by signs, symptoms or complications.

Internal hemorrhoids are cavities containing blood, having approx. 2-15 mm diameter, separated by a venous endothelium. At this level are met arterio-venous anastomoses, which cause cavernous type structures and direct anastomosis between vessels and the anal canal mucosa. These vascular structures are held in position through the Parks ligament and through some muscular-conjunctive fibers that form a genuine suspensory ligament connected to the anal mucosa, to the internal sphincter and to the longitudinal muscle layer.

Hemorrhoidal tissue (vascular structures—muscular-conjunctive fibers) does not form a continuous ring, being interrupted here and there by connective tissue and being agglomerate especially in three main areas: the left side, right front and right rear.

The rectum and the anal canal are irrigated by three arteries, between which there are extensive anastomoses: superior rectal, middle rectal and lower rectal. The arterial flow of internal hemorrhoids is essentially originated in submucosa and comes from the superior rectal artery.

Internal hemorrhoids drain into superior rectal vein—tributary to the portal vein and middle rectal vein—tributary to the inferior vena cava. Thus, the internal hemorrhoids are porto-caval anatomical anastomosis but never get the meaning of other porto-caval anastomosis in pathologic situations.

The term “hemorrhoids” comes from the ancient Greek (haima=blood, rhoos=leakage) and therefore could be translated as “blood loss”, which is related to the most significant symptom of this condition. Classically, hemorrhoids are defined as varicoseous dilatations of anorectal veins.

Under the influence of various factors, there is an alteration of structure and the trophic hemorrhoidal plexus of the vein wall, which has as consequence the changing of the physiological venous dilatations into pathological ones, as the number, size and confluences are concerned. Thus, disturbances occur in the local circulation, mainly the venous stasis which, on one hand, worsens secondarily the vein wall injuries by emphasizing ectasia and, on the other hand, promotes bleeding and thrombosis with subsequent determination of signs and complications of hemorrhoidal disease.

Factors responsible for the alteration of the wall trophicity of the hemorrhoidal plexus veins are:
local inflammation (cryptitis, suppurations)—by fostering a chronic phlebitis process that alters the collagen and elastic fibers, leading to the emergence of phlebectasia;
endocrine factor—pregnancy causes hemorrhoids, by temporarily inhibiting the posterior pituitary, whose hormone may play a role in maintaining the venous tone, along with the action of “weakening” of the pelvic connective tissues induced by the action of relaxin (hormone produced during pregnancy, in order to obtain the pelvic tissue laxity preparing to deliver);
genetic factor—may constitutionally, familiarly, even racially cause hemorrhoids by venous dysplasia, which explains the association with varicose veins, varicocele, flat foot, hernias and the aggregation of hemorrhoidal disease in some families.

On the other hand, the local venous stasis may be maintained and exacerbated by a number of factors that hinder the return circulation:—extended orthostatism;—sedentarism;—physical effort;—the absence of venous valves at this level;—difficult defecation;—pregnancy;—compressions on return circulation (rectal and pelvic tumors, portal hypertension etc.);—inflammatory and infectious colopathies diseases.

Although the source of bleeding in hemorrhoidal disease should be venous, actually the lost blood is usually light red arterial blood, well oxygenated, and its source is the submucosal arterio-capillary network, impaired by local inflammatory phenomena, venous stasis, local microtraumas caused by faeces emission or chemical irritation by ingestion of alcohol and spices.

Internal physiological hemorrhoidal dilatations are fixed and held in position by a fibro-musculo-elastic tissue which tends to degenerate with age.

The natural evolution of hemorrhoidal disease include intermittent periods of “quiet” periods in which characteristic symptoms are present and can be explained by supra-adding occasional inflammatory phenomena.

There is no single theory that could explain the essence of hemorrhoids transformation from physiological into pathological. However, the degradation of suspensory ligament is now considered the essential premise of the emergence and development of hemorrhoidal disease, other factors contributing to continuous or intermittent worsening of degenerative lesions and symptoms.

### BACKGROUND ART

On these considerations are based instrumental, non-surgical treatments, whose purpose is not the compulsory removal of hemorrhoidal mass, but a reattachment of them into physiological position.

Etiologically, hemorrhoids can be divided into:—symptomatic—the expression of a hyperpression in the port system or pelvis;—idiopathic—the expression of the described hemorrhoidal land.

Clinically, hemorrhoids are classified into:—bleeding;—procident (prolabated);—painful;—with leaks (serous or purulent).

Anatomo-clinically, hemorrhoids are divided into:
internal—submucosal or supra-sphincterial, developed on the superior hemorrhoidal plexus, which constitutes the principal premise of the disease and which, depending on the degree of their development and of trans-anal prolabation, may vary from Ito IV;
external—subcutanated or infra-sphincterial, developed on the inferior hemorrhoidal plexus, much more rare and challenged today by most authors.

Hemorrhoidal disease (identical to the term “internal hemorrhoids”) generally occurs through five symptoms: bleeding, pain, prolapse, leaks, itching, discussed in the chapter on.

Bleeding is typically caused by defecation, following it as varying amounts of bright red blood, enveloping but not mixed with stool. 10% of patients with hemorrhoids do not show any symptoms; hemorrhoids are generally not so dangerous as by bleeding to threat life (except in special cases).

Pain: in general, except crises, internal hemorrhoids are not painful, the most discomfort may occur during prolapse; pain appeared out of crisis often evoques a pathological association (anal fissure, abscess).

Prolapse: met for 50% of patients, is due to the sliding of the bulky hemorrhoidal mass toward the anal oriffice and outwards, through the loose submucosa also decollated of mucosa.

Although there is no anatomo-clinical correlation between the condition degree and the severity of symptoms, for teaching purpose and for a suitable treatment option, internal hemorrhoids were classified into 4 grades of disease:—Grade I: non-procident;—Grade II: prolapsing and spontaneously reducible;—Grade III: prolapsing, but only manually reducible;—Grade IV: prolapsing non-reducible.

At grades III and IV bleeding or secreting exulcerations may be observed, due to congestion of the mucous membrane at the contact with linen, alternating with areas of keratinization.

In the case of hemorrhoidal anal prolapse (grade IV), it is viewed tumorette with mucosal folds radially oriented, which really sets it apart from the true anorectal prolapse, where these folds have a circumferential orientation.

Leaks and itching are the expression of inflammation of anal mucosa (serous leakage) and a consequence of glandular hypersecretion which may become purulent if it is superinfected.

Permanent moisture of the perianal skin increases skin irritation and eczematides will generate itching.

Regarding the location of hemorrhoids, the three main classical sites are losing their actuality, whereas modern studies have shown that internal hemorrhoids always appear circularily, as are arranged the Duret ridges at Morgagni ampoules of origin.

External hemorrhoids can cause symptoms in two situations:

External hemorrhoidal thrombosis (a very common situation), consisting of the occurrence of nonreducible, painful, blue-violet swelling, located on the anus edge and covered by teguments. Spontaneous evolution goes toward the clot resorption after 7-14 days. “External” hemorrhoids are generally challenged as being on their own, these thrombosis representing post-traumatic hematoma (constipation, exercise) in modern sense, actually as an expression of “decompensation” of the internal hemorrhoids;

Hemorrhoidal marisca—remaining skin folds after clots absorption, from the hemorrhoidal thrombosis, are not symptomatic and do not represent a pathological change, but no more than a unaesthetic sequel, except the cases they are bulky, painful, or prevent local hygiene.

The diagnosis of hemorrhoidal disease is always a clear and simple diagnosis that does not require sophisticated means. More important, however, is the well motivated elimination of possible concomitances, sometimes more serious ones, with overlapping symptoms (cancer, ulcerative colitis etc.).

External hemorrhoids do not fall in question, except the thrombosis or “unaesthetic” sequelae.

Nowadays, symptomatic hemorrhoids are denied as an entity and considered random coincidences and managed in the context of pathology.

Evolutively, internal hemorrhoids are undergoing a series of stages, falling within the 4 degrees.

Against this background can graft congestive hemorrhoid spurts, painful, which can be repeated, the evolution being aggravated either by septic phenomena, anemia or psychic phenomena (neurasthenia, anxiety etc.):
supra-hemorrhoidal thrombophlebitis—represents the location of thrombophlebitic phenomena on the related branches of submucosal venous plexus, an extension of the ordinary hemorrhoidal thrombosis;
bleeding complications—are represented by iron deficiency anemia, rarely severe; in the cases with rare bleedings there is no systemic response;
suppurative complications—in the form of anal and perianal abscesses, represents the spread of an infection from suppurated hemorrhoids;
other complications—hemorrhoidal pseudopolyps, anal fissure, mental disorders.

Currently, surgery is no longer the hemorrhoidal disease therapy, except the cases of failure of medical and instrumental treatment. The treatment goal is to influence the mobilization of hemorrhoids and mucosal fragility, and not necessary the removal of those venous dilatations. Avoiding sedentarism, spices, alcohol can reduce the action of predisposing factors.

The goal of instrumental treatment is the symptomathology cropping by:—removing most of hemorrhoidal mass,—maintaining an adequate venous dilatation in order to ensure perfect anal continence, and—setting the dilated vein to prevent prolapse and recurrences.

Starting from a number of disadvantages and complications of surgery, were also devised a number of methods capable of achieving these goals.

All these methods are considered non-surgical and must be the first line treatment for all hemorrhoids unresponsive to conservative treatment. The joint effect of these methods is to produce a limited area of sclerosis on the apex of the mobile mass of hemorrhoidal tissue, resulting in the wound healing of this mucosal and submucosal area adhesion to deep muscle blanket. Therefore, the aim is not to destroy the hemorrhoid, but especially to reposition it inside the anal canal, to reduce its size and its attachment to the wall by a fibrous scar.

These methods solve the symptoms causes, hemorrhoids resuming their physiological role, providing protection against mechanical action and ensuring fine gas continence.

Currently are used: rubber band ligation; infrared photocoagulation or photocoagulation (laser); sclerotherapy; cryotherapy; hemorrhoidal artery ligation (HAL); galvanic hemorrhoidolysis; monopolar or bipolar diathermocoagulation.

In all these processes, cutting, dissection, hemostasis can be performed by classic scalpel, electric scalpel, with ultrasounds, radiofrequency or laser. All these procedures involve the possibility of occurrence of specific anesthetic and surgical complications and of post-surgical sequelae, sometimes powerful or disabling ones, with a high rate of relapse.

These considerations led to the idea of abandoning them, especially along the development of instrumental techniques.

Sometimes medicinal treatment is indicated in the case of hemorrhoids grade I or II, with intermittent symptoms, for debilitated patients, consecutively other underlying conditions, especially in the treatment of acute thrombophlebitic crisis during which aggressive treatments are usually proscribed.

It is actually a symptomatic treatment, ensuring the mitigation or providing temporary disappearance of symptoms until the curative treatment (non-surgical or surgical) which will be used, given the inexorable progress of the disease.

There is a very large number of topical agents, composed of various combinations of antiinflammatory agents (cortisone), local anesthetics, lubricants, “vasculo-trophic” derivatives etc.

But topical agents act primarily through the anal canal lubrication, facilitating evacuation of faeces. They have the advantage of being applied at the place of suffering, which creates some psychological comfort to patients. In fact, there is no serious study demonstrating their effectiveness. Furthermore, creams and ointments produced, especially in the case of long use, allergic dermatitis, and suppositories even appear illogical because they act intra-rectally and may micro-traumatize the anal canal at the time of inclusion.

Veno-trophics—flavonoid derivatives, represent the only medication with proven action on hemorrhoidal disease. Flavonoid derivatives decrease capillary fragility and have anti-inflammatory effect, preventing leukocyte adhesion, increase venous tone and activate microcirculation.

It represents the main treatment option in hemorrhoidal crisis. In the long run, however, do not represent an etiological treatment of this condition, it can only prevent or rarefy the possible thrombophlebitic recurrency.

The most common concoction is Detralex.

The document CA1133831 shows a composition against dental periodontosis and haiiotis, including calcium carbonate, betel, ginger, pepper, salt, myrobalan and camphor, and in the document RU2137464 CI it is presented a toothpaste with an effect combating gingivitis, inflammation, and stimulating trophic and regenerative processes, which has into composition mustard seeds, methyl salicylate, sea salt, calcium carbonate, glycerol, thymol, camphor and menthol. Also, the document WO9605797 A discloses a skin ointment with nutritive and protective properties, based on fat (30-70%) which also includes: vitamin A, salicylic acid, camphor 2-4%, aminobutyric acid, dopamine, pancreatin, ascorbic acid, calcium pantothenate, vitamin D2, and water.

### TECHNICAL PROBLEM

The technical problem solved by the invention is to obtain an effective ointment for the treatment of hemorrhoidal disease against internal hemorrhoids, avoiding the need for surgery in the long term, being also cheap and affordable.

### SOLUTION TO PROBLEM

According to the invention, the ointment for the treatment of hemorrhoidal disease mainly intended to the treatment of internal hemorrhoids, grades I and II, solves this technical problem by having into the composition, in weight percent, 38-44% adeps suillus axungia, 18-23% lanolin, 18-23% white vaseline, 2-3% camphor; 7-12% calcium carbonate, 0.2-0.5% eucalyptus oil, 1.5-2% ethanol (ethyl alcohol) and 0.1-0.5% usual preservative.

The active components thereof camphor and eucalyptus oil in combination with calcium carbonate and the base ointment components adeps suillus (axungia), lanolin and white vaseline, in the declared percentages, their mutually reinforcing effects, producing through synergy a final effect at least equivalent with known products, but without the unwanted side effects.

### ADVANTAGEOUS EFFECTS OF INVENTION

The ointment according to the invention has the advantage of eliminating the need for surgery as the treatment of hemorrhoidal disease by being efficient and easy to administer, well tolerated by patients, without side effects, with good efficacy in all forms hemorrhoidal disease.

### DESCRIPTION OF EMBODIMENTS

The project for manufacturing and testing of ointment was divided into two phases (Table 1): a design phase and a test phase of the new product (testing performed at the Faculty of Medicine and Pharmacy of the University “Dunarea de Jos”).

TABLE 1
Project Phases

| Nr. Crt. | Phase name |
|---|---|
| Phase I | Biochemical characterization, microbiological and functional |
| | product |
| | Camphor: is a substance made from the resin of the tree terpenes |
| | group “Cinnamomum Camphora”. It has antipruritic action; |
| | antimicrobial; inflammatory and painkiller, and not least |
| | stimulates blood circulation. |
| | Calcium carbonate: derivative of calcium with trophic effect |
| | upon venous vessel walls; increases tonicity of the venous wall |
| | leading to the retraction of venous dilatations. |
| | Adeps suillus (axungia): it is absorbed in a very small |
| | percentage, forming a protective layer on the vessel wall, |
| | reducing traumatic activity and facilitating the effect of |
| | the other components at this level. |
| | Eucalyptus oil: is a substance from the terpenes group. It has |
| | antiinflammatory and analgesic action when applied topically. |
| Phase II | Performing clinical trial |
| | Selection of the group of patients. Clinical evaluation of patients. |
| | Paraclinical evaluation of patients, in order to exclude possible |
| | pathologies, caused or contributing to hemorrhoidal disease. |
| | Applying the treatment |
| | Subjectively and objectively monitoring patients, after treatment. |
| | Teaching results. |

The set percentage composition of the ointment is shown in the Table 2 below:

TABLE 2
The percentage composition of ointment:
percentage limits and embodiment

| | Quantity: | Example: Quantity: |
|---|---|---|
| | x g/100 g | y g/100 g |
| Name of the raw material | ointment | ointment |
| Camphor | (2-3) | 2.5 |
| Calcium carbonate | (7-12) | 10 |
| Lanolin | (18-23) | 21.3 |
| White Vaseline | (18-23) | 21.3 |
| Adeps suillus (Axungia) | (38-44) | 42.6 |
| Eucalyptus oil | (0.2-0.5) | 0.3 |
| Ethanol (ethyl alcohol) | (1.5-2) | 1.8 |
| Usual Preservative | (0.1-0.5) | 0.2 |

The obtaining of the ointment according to the invention is classical. In principle, ointment base components are heated, mixed and homogenized. Then incorporate other ingredients in the composition at a time with mixing after each addition of a constituent of the composition. Camphor is added to the composition after dissolving in ethanol.

The camphor used is a crystalline mass, translucent or white crystalline powder with characteristic odor and pungent taste at first, slightly bitter, then refreshing. It volatilises at room temperature. It burns with fuligineous flame, without leaving residue. It lightly sprays in the presence of alcohol, chloroform or ether. It is very slightly soluble in alcohol, chloroform, ether, and oil of turpentine, readily soluble in liquid paraffin, fatty oils, and oils, water-soluble (slightly soluble in water heated to 80° C.), insoluble in glycerol.

On medical purpose it is used as a local topic in combating itching and as antimicrobial agent and is already included in many products under different patented chemical compositions.

The Eucalyptus oil (eucalyptol) is a natural organic compound, liquid, colorless. Is a cyclic ether and a monoterpenoid. It is found in camphor laurel, bay leaves, tea tree, wormwood, basil, rosemary, sage, cannabis sativa leaves and other herbs. Eucalyptol with 99.6-99.8% purity can be obtained in large quantities by fractional distillation of eucalyptus oil. Eucalytpol has a fresh camphor-like smell, as well as a spicy, refreshing taste. It is insoluble in water but miscible with ether, ethanol and chloroform.

Due to its aroma and pleasant taste, it is used in flavorings, perfumes and cosmetics. Eucalyptol oil is used as flavoring (at low concentrations—0.002%) in various products, including pastries, confectionery, meat products and beverages. Although it can be used in medicine as an ingredient in very small doses, though it is toxic if taken in doses higher than normal; in high doses can be dangerous by ingestion, skin contact or inhalation, it may have serious effects on behavior, respiratory and nervous system. In large doses, patients may experience headache, impaired general condition, nasal obstruction and nasal discharge. Since 1994 is used as an additive for cigarettes. Also is an ingredient in many brands of mouthwash and cough. Eucalyptol is used as an insecticide and insect repellent.

Recent studies in 2000-2004 revealed that the eucalyptol is useful as inhibiting hypersecretion of mucus in asthma, suppressing the production of arachidonic acid, in the suppression of cytokines in human monocyte transformation for the treatment of nonpurulent rhinosinusitis. In a 2002 study it was shown to destroy leukemia cells in two cultured cell lines. A 2000 study found that eucalyptol reduces inflammation and pain when applied topically.

The Calcium Carbonate is a common substance, spread in nature under mineral forms: calcite, aragonite, vaterite and under organic forms—bones, teeth, shells, corals and crustaceans crust; in rocks is in the form of limestone, which is almost pure and Dolomite varient, which are a mixture of calcium and magnesium. It is found in rocks all over the world, and is the main component of shells of marine organisms, snails, coal balls, beads and eggshells. Calcium carbonate is an active ingredient of agricultural lime, and is created when the Ca ions in hard water react with the calcium carbonate.

Calcium carbonate contains at least 98.5% and not more than 100.5% CaCO3. It is used in the form of a fine powder, microcrystalline, white, odorless and tasteless.

It is usually used as a medicine, as a calcium supplement or as antacid in the treatment of osteoporosis, as filler in the pharmaceutical industry etc. It can be used as phosphate binder for the treatment of hyperphosphatemia (primarily in patients with chronic renal failure). It is also used in the pharmaceutical industry as inert filler for tablets and other pharmaceutical products. Calcium carbonate is used in the production of toothpaste. In the food industry it is used as acidity regulator, anti-caking agent, stabilizer, coloring or curing agent in many canned or bottled vegetable products.

Excess calcium in food supplements and calcium-rich diets, may cause the alkali milk syndrome, with severe toxicity, which can be fatal. Excessive calcium intake may lead to hypercalcemy, complication manifested by vomiting, abdominal pain and altered mental status.

Calcium carbonate has a trophic effect on venous vessel walls, increases venous wall tonicity, leading to retraction of venous dilatations.

Usual preservative—is a preservative that can be easily chosen by the skilled person on the basis of general knowledge about the usual preservative for adding in the composition of pharmaceutical ointments with topical application to mucous membranes and skin injured, they are preservatives without irritating when applied to targeted areas.

For example, the skilled person could choose a preservative or mixture of preservatives commercially available in parabens class.

Parabens, p-hydroxygenzoic acid esters are used alone or in combination, to exert their effect on fungi, yeasts, molds, and on bacteria. As antimicrobial preservatives, parabens have been proposed for drugs and nutrition since 1900, they have over 100 years of use and have been proven to be very effective antimicrobial agents. Parabens can have multiple biological actions. These antimicrobial preservatives exhibits many of the criteria of an ideal preservative, such as broad spectrum, safety of use: are relatively non-irritating, non-sensitized, low toxicity, are stable in a wide range of pH=4-8 (by other authors 7-9). The main p-hydrozybenzoic acid esters used are: methyl-, ethyl-, propyl-, and butyl paraben. Parabens are stable in air, water resistant to hydrolysis in water (cold and hot) and in acidic solutions.

### EXAMPLES (CLINICAL TRIAL)

The clinical trial was done on a group of patients diagnosed with hemorrhoidal disease, 36 people over a period of 90 days (01.02.2013-01.05.2013)

Medical tests for monitoring the patients included in the study, performed at the start of a clinical trial to assess the overall health status were: CBC; ESR; GGT TGO TGP abdominal ecography;
the average age was 46.8 years, with a predominance of hemorrhoidal disease between 40 and 60 years (21 cases representing 58.33%), with a peak age of incidence for the decade 50-60 years (11 cases, representing 30, 55%);
the incidence by sex showed a predominance of females, with 25 of the 36 cases, representing a rate of 69.45%, while males accounted for only a percentage of 30.55%, which is only 11 males;
out of the 36 patients, a total of 27 patients had recurrence of hemorrhoidal disease (75%), while only 9 patients were free of hemorrhoidal history as the first manifestation of the disease (25%), highlighting the chronic and recurrent nature of the disease, known as evolution by relapses of acute hemorrhoidal disease;
a total of 19 patients (52.78%) had a family history, finding that younger people showed mostly family history;
the predominant symptom was pain (72.22%), followed by anal itching, 22 patients (61.11%) and rectal—22 patients (61.11%) and the presence of wet anus—12 patients (33.33%);
it was found that at the time of initial, examination, pre-treatment, most patients had grade II hemorrhoids—26 patients (72.22%), followed by those with hemorrhoids grade I—6 patients (16.66%) and those with hemorrhoids grade III—4 patients (11.12%);
regarding the therapeutic results, as reported by patients, it has been revealed that 88.46% reported a significant pain mitigation up to the complete disappearance of it, 72.22% reported a reduction up to alleviate itching, 88.88% of patients presenting acute inflammation of hemorrhoidal disease have reported significant reduction of swelling and local pain, and 50% of patients reported rectal bleeding stopped;
the treatment was considered effective by 35 patients (97.22%), noting reducing symptoms and improving quality of life, while only one patient with internal hemorrhoids grade III associated with external hemorrhoids, a hard compliant patient, argued that treatment is ineffective (2.78%);
no patient presented side effects or intolerance product;
none contraindications emerged, the only deducted counter-indication represented by the patient intolerance to one of the constituents of the product;
a total of 26 patients (72.22%) showed gradual remission of the disease, 7 patients (19.44%) showed a significant improvement in symptoms, requiring long-term treatment, and only 3 patients (8.34%) were stationary;

The product addresses hemorrhoidal disease of all grades, yielding favorable results regardless of the grade therefor.

### PATENT LITERATURE

PTL1: CA1133831 A: Dental powder with natural ingredients

PTL2: RU2137464 CI: Tooth Paste

PTL3: W09605797 A: Agent for external application with nutrient and protective properties

1. Ointment for the treatment of hemorrhoidal disease, based on animal fat, in particular for the treatment of internal hemorroids grades I and II, characterized in that it comprises, in weight percent, 38-44% adeps suillus axungia, 18-23% lanolin, 18-23% white vaseline, 2-3% camphor; 7-12% calcium carbonate, 0.2-0.5% eucalyptus oil, 1.5-2% ethanol (ethyl alcohol) and 0.1-0.5% usual preservative.
